(19) United States
(12) Patent Application Publication
(10) Pub. No.: US 2026/0199088 A1
(43) Pub. Date: Jul. 16, 2026

(54) PROSTHETIC MEDICAL DEVICE DELIVERY SYSTEM

(71) Applicant: Edwards Lifesciences Corporation, Irvine, CA (US)

(72) Inventor: Kurt Kelly Reed, Costa Mesa, CA (US)

(21) Appl. No.: 19/561,960

(22) Filed: Mar. 10, 2026

Related U.S. Application Data
(63) Continuation of application No. PCT/US2024/046174, filed on Sep. 11, 2024.
(60) Provisional application No. 63/620,990, filed on Jan. 15, 2024.
(60) Provisional application No. 63/582,463, filed on Sep. 13, 2023.

(51) Int. Cl. A61F 2/24

(52) U.S. Cl. CPC A61F 2/2436

(57) ABSTRACT

A delivery apparatus can include a shaft extending in an axial direction of the delivery apparatus. The shaft can include an outer surface and a proximal end portion. A support member can be disposed circumferentially around the proximal end portion of the shaft and can include an inner surface and a sealing channel extending radially outwardly from the inner surface. A sealing member can be radially disposed between the shaft and the support member and can be at least partially disposed within the sealing channel. The sealing member can contact the outer surface of the shaft and the inner surface of the support member.

### CROSS REFERENCE TO RELATED APPLICATIONS

This application is a continuation of PCT Patent Application No. PCT/US2024/046174 filed on September 11, 2024, which claims the benefit of U.S. Provisional Application No. 63/620,990, filed on January 15, 2024, and U.S. Provisional Application No. 63/582,463, filed on September 13, 2023, the entire disclosures of which are incorporated by reference herein.

### FIELD

The present disclosure related to delivery systems for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

A guide catheter (which can also be referred to as a guide sheath) can be used for introducing a delivery apparatus, such as the prosthetic heart valve delivery apparatus described above, into the patient’s vasculature. The guide catheter can include an elongated shaft that is inserted into the vasculature and a handle that remains outside the patient and can be used to manipulate the shaft. The delivery apparatus can be pushed through a main lumen of the guide catheter to help navigate the delivery apparatus to a target implantation site within the patient.

### SUMMARY

Described herein are guide catheters, delivery apparatuses, methods for forming guide catheters and/or delivery apparatus, and methods for implanting prosthetic heart valves. The guide catheters, delivery apparatuses, and methods can, for example, provide for improved sealing within a guide catheter during an implantation procedure to further improve the reliability and/or robustness of the guide catheter and/or implantation procedure. As such, the devices and methods disclosed herein can, among other things, overcome one or more of the deficiencies of typical prosthetic heart valves, docking devices and associated delivery apparatuses.

A delivery apparatus can comprise a handle and one or more shafts coupled to the handle.

In some examples, the shaft can comprise an outer surface and a proximal end portion.

In some examples, the delivery apparatus can further comprise a support member disposed circumferentially around the proximal end portion of the shaft.

In some examples, the support member can comprise an inner surface and a sealing channel extending radially outwardly from the inner surface.

In some examples, the delivery apparatus can further comprise a sealing member radially disposed between the shaft and the support member and at least partially disposed within the sealing channel.

In some examples, the sealing member can contact the outer surface of the shaft and the inner surface of the support member.

In some examples, the sealing member can comprise a locking element.

In some examples, the locking element can extend in a radially outwards-facing direction.

In some examples, the locking element can taper in a radially inwards-facing direction.

In some examples, the sealing member can occupy the entirety of the sealing channel.

In some examples, the sealing member can comprise a chamfered surface tapering in a distal direction of the delivery apparatus.

In some examples, the sealing member can comprise a proximal annular portion and a distal annular portion.

In some examples, the proximal annular portion can have a larger diameter than the distal annular portion.

In some examples, the sealing member can be a molded component.

In some examples, the sealing member is not chemically bonded to either the shaft or the support member.

In some examples, the sealing channel can further comprise a circumferential aperture formed on an outer surface of the support member.

In some examples, the sealing channel can comprise a tapered portion that tapers in a radially inwards-facing direction.

In some examples, the sealing member can comprise a locking element, and wherein the tapered portion can be configured to receive the locking element therein.

In some examples, the locking element can be tapered, and wherein the taper of the locking element can be complementary to the taper of the tapered portion.

In one example, a delivery apparatus can comprise a shaft extending in an axial direction of the delivery apparatus, a support member disposed circumferentially around the proximal end portion of the shaft, and a sealing member radially disposed between the shaft and the support member. The shaft can comprise an outer surface and a proximal end portion. The support member can comprise an inner surface and a sealing channel extending radially outwardly from the inner surface. The sealing member can be at least partially disposed within the sealing channel. The sealing member can contact the outer surface of the shaft and the inner surface of the support member.

In one example, a delivery apparatus can comprise a handle, a handle lumen extending through the axial length of the handle, a shaft extending distally through at least a portion of the handle lumen and axially aligned with the handle lumen, a support member circumferentially surrounding the shaft, and a sealing member. The shaft can comprise a shaft lumen extending through the axial length of the shaft and a proximal end portion. The support member can comprise a sealing channel extending from an inner surface of the support member in a radially outwards-facing direction. The sealing member can comprise an annular body contacting an outer surface of the shaft at the proximal end portion.

In one example, a method of forming a delivery apparatus can comprise forming a support member, wherein the support member can comprise a support member lumen extending through the axial length of the support member and a sealing channel extending from an inner surface of the support member. The method can further comprise inserting a first core pin into a first end of the support member lumen, axially aligning a first end portion of the first core pin with the sealing channel, inserting a second core pin into a second end of the support member lumen, axially aligning a second end portion of the second core pin with the first end portion of the first core pin; and injecting a mold material into the sealing channel to form a sealing member.

In some examples, a method comprises one or more of the components recited in Examples 1-23 below.

The above method(s) can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a stage in an exemplary mitral valve replacement procedure where a guide catheter and a guidewire are inserted into a blood vessel of a patient and navigated through the blood vessel and into a heart of the patient, towards a native mitral valve of the heart.

FIG. 2A schematically illustrates another stage in the exemplary mitral valve replacement procedure where a docking device delivery apparatus extending through the guide catheter is implanting a docking device for a prosthetic heart valve at the native mitral valve.

FIG. 2B schematically illustrates another stage in the exemplary mitral valve replacement procedure where the docking device of FIG. 2A is fully implanted at the native mitral valve of the patient and the docking device delivery apparatus has been removed from the patient.

FIG. 3A schematically illustrates another stage in the exemplary mitral valve replacement procedure where a prosthetic heart valve delivery apparatus extending through the guide catheter is implanting a prosthetic heart valve in the implanted docking device at the native mitral valve.

FIG. 3B schematically illustrates another stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates another stage in the exemplary mitral valve replacement procedure where the guide catheter and the guidewire have been removed from the patient.

FIG. 5 is a side view of a guide catheter configured to receive a delivery apparatus and/or a tool and guide the delivery apparatus and/or the tool through a portion of a patient’s vasculature, according to one example.

FIG. 6 is a side view of a sealing housing portion of the guide catheter of FIG. 5, according to one example.

FIG. 7 is a cross-sectional view of a proximal end portion of a guide catheter, according to one example.

FIG. 8 is a cross-sectional view of a proximal end portion of a guide catheter, according to one example.

FIG. 9 is a cross-sectional view of a proximal end portion of a guide catheter, according to one example.

FIG. 10 is a cross-sectional view a stage of the guide catheter fabrication process in which a guide catheter support member is formed, according to one example.

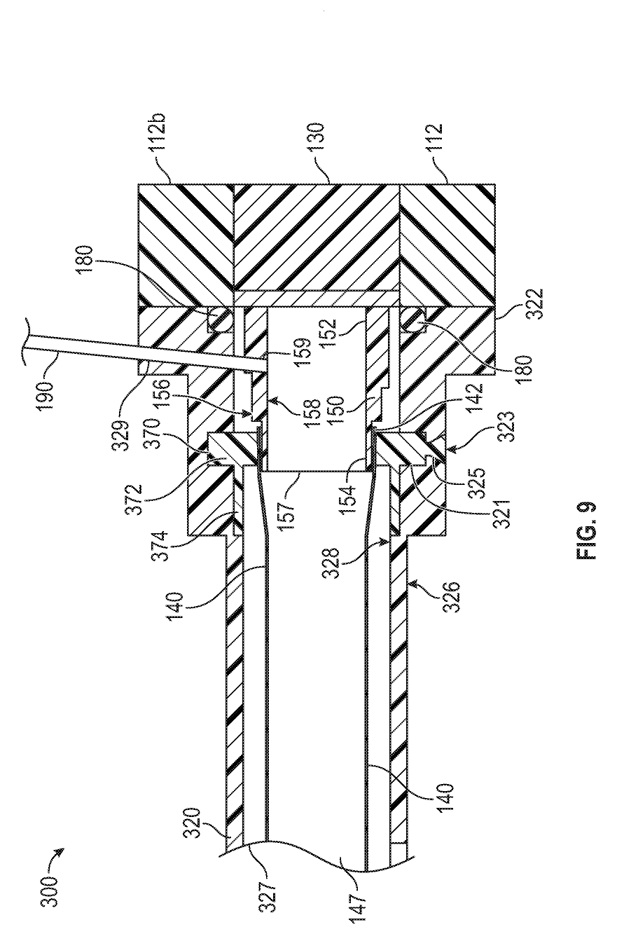

FIG. 9 illustrates a stage in an exemplary guide catheter fabrication process where two core pins are inserted into a guide catheter support member.

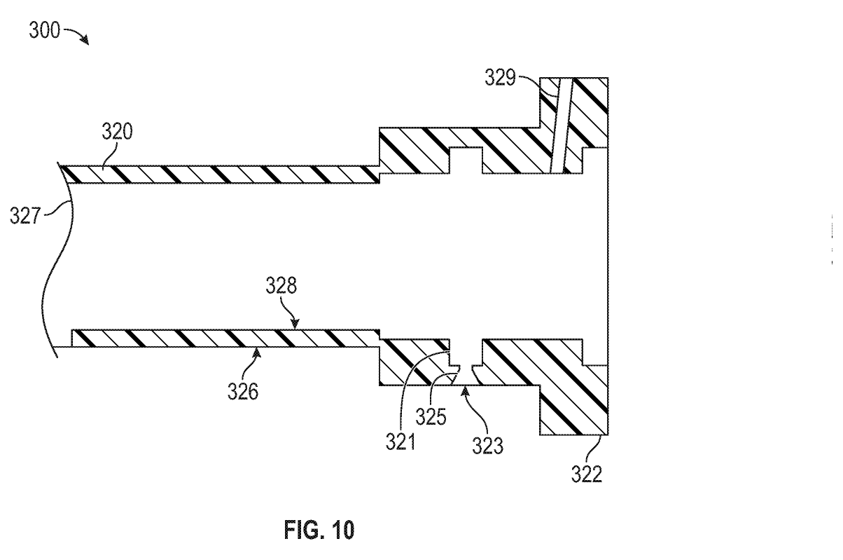

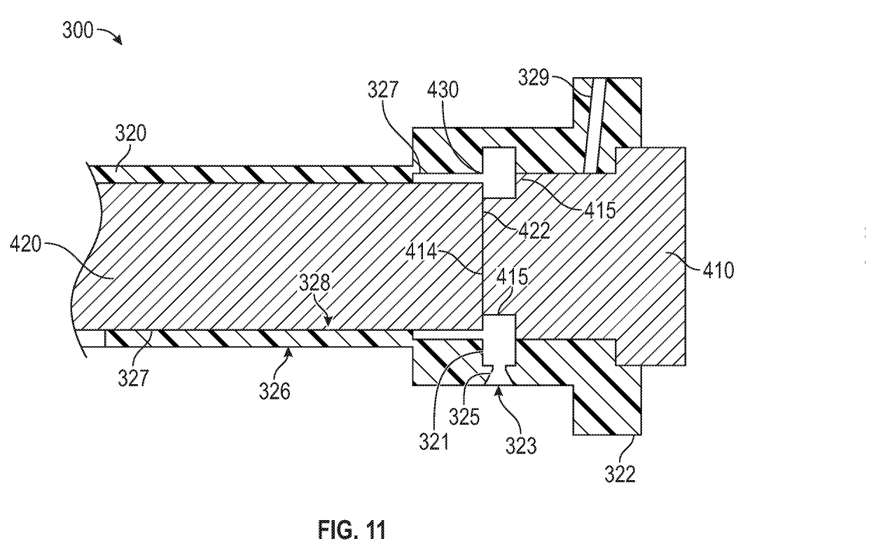

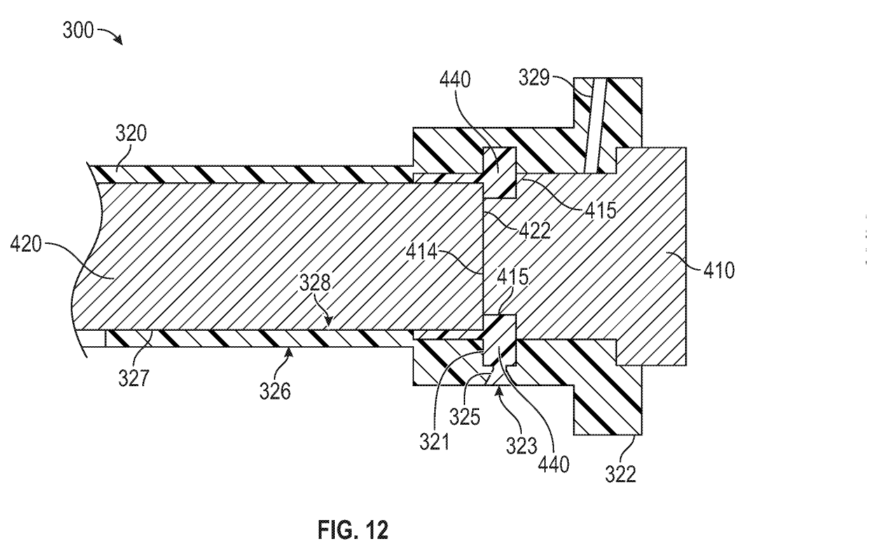

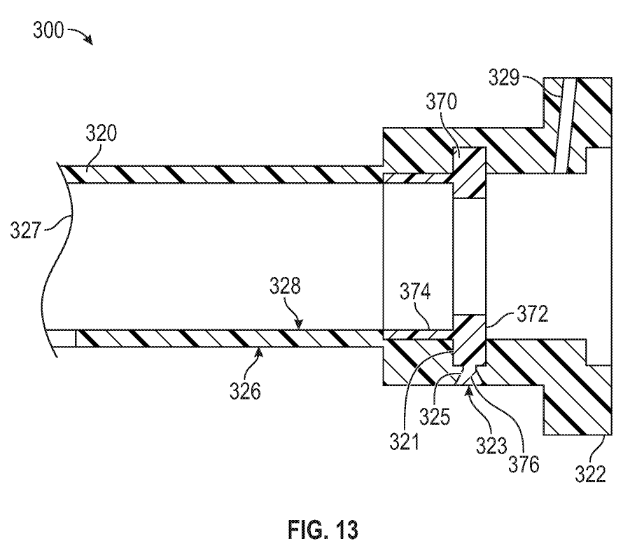

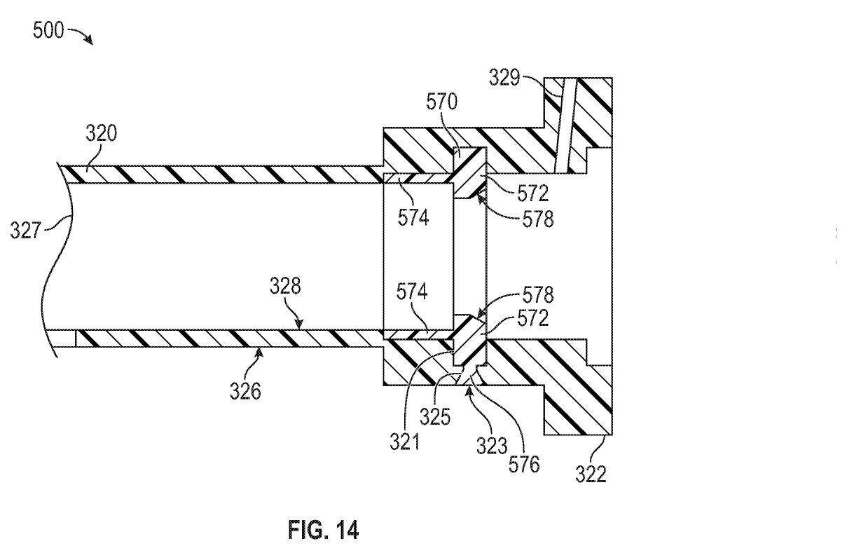

FIG. 10 illustrates another stage in the exemplary guide catheter fabrication process where mold material is injected to form a sealing member.

FIG. 11 is a cross-sectional view a stage of a guide catheter fabrication process in which first and second core pins and are inserted into a guide catheter support member lumen, according to one example.

FIG. 12 is a cross-sectional view a stage of a guide catheter fabrication process in which mold material is injected into a sealing member cavity, according to one example.

FIG. 13 is a cross-sectional view a stage of a guide catheter fabrication process in which a first core pin and a second core pin are removed from a guide catheter support member lumen, according to one example.

FIG. 14 is a cross-sectional view of a proximal end portion of a guide catheter, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (such as out of the patient’s body), while distal motion of the device is motion of the device away from the user and toward the implantation site (such as into the patient’s body). The terms “longitudinal” and “axial” refer to an axis extending in the proximal and distal directions, unless otherwise expressly defined.

The terms “lateral” and “radial” refer to an axis perpendicular to the longitudinal axis. When referring to a “lateral” direction with respect to a stabilizer assembly for a docking device delivery system, the term “lateral” refers to an axis that perpendicular to the longitudinal axis and parallel with a plane defined by a stabilizer track of the stabilizer assembly.

As used herein, “e.g.” means “for example,” and “i.e.” means “that is.”

### Introduction to the Disclosed Technology

Disclosed herein are examples of a delivery system that can be used to help navigate a subject’s vasculature to deliver a prosthetic medical device (such as a prosthetic heart valve and/or a docking device used in conjunction with a prosthetic heart valve), tools, agents, or other therapy to a target implantation site within the body of the subject.

In connection therewith, various features are described herein that, in some examples, can provide for better sealing within the delivery system to better improve the positioning of the prosthetic.

### Examples of the Disclosed Technology

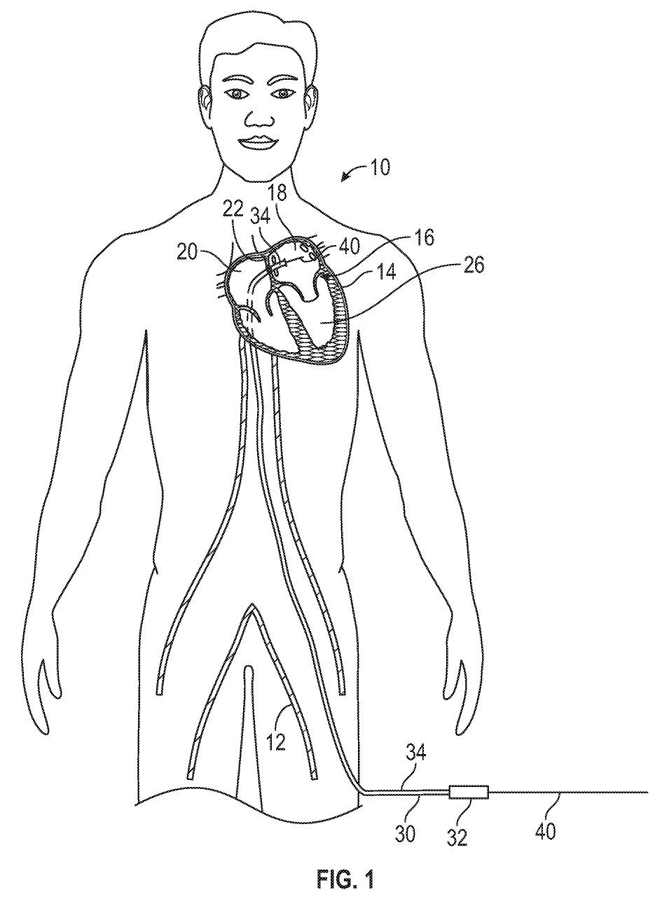

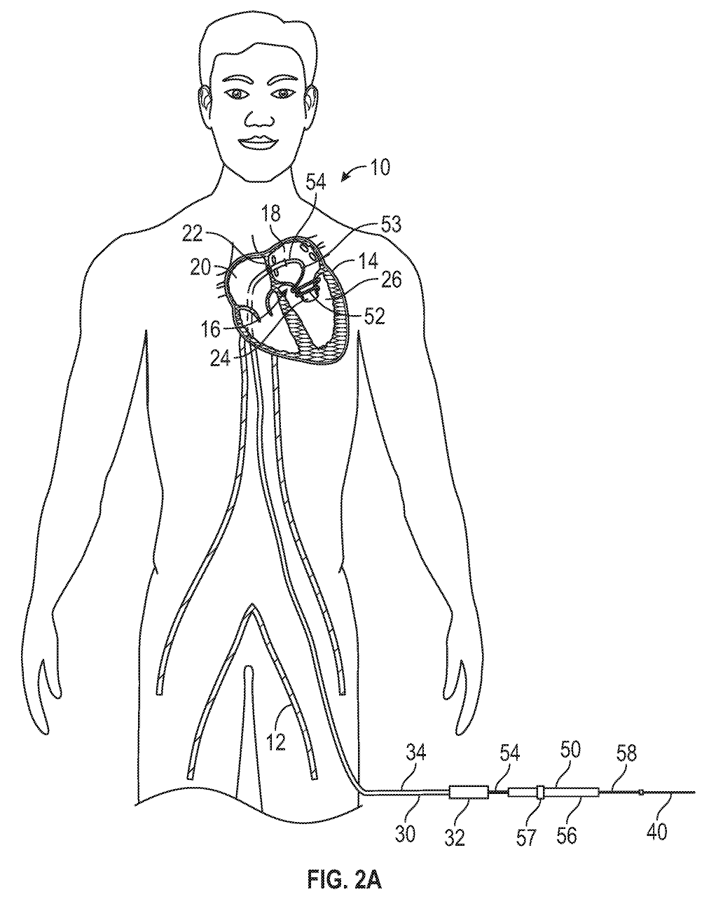

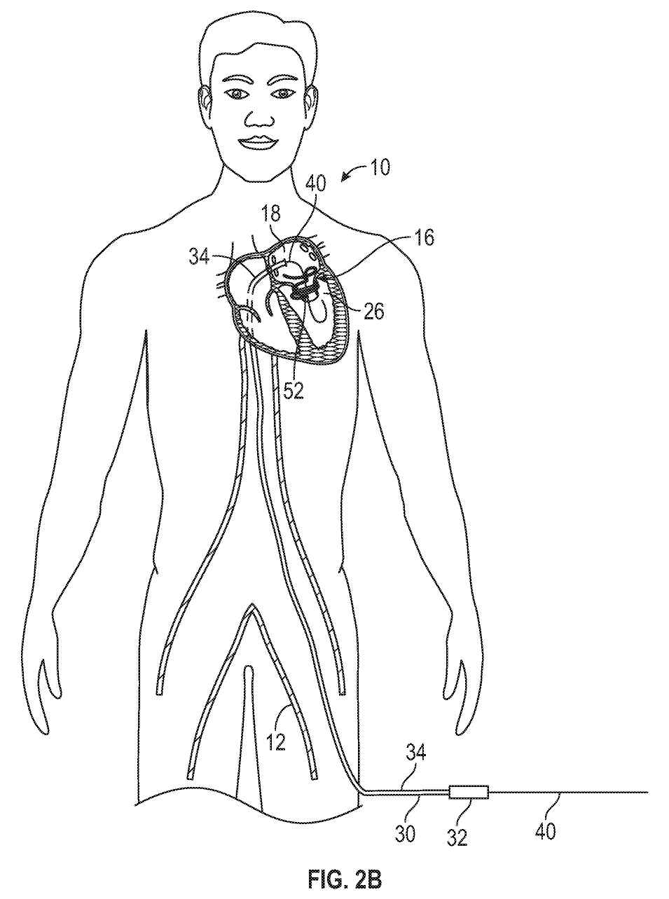

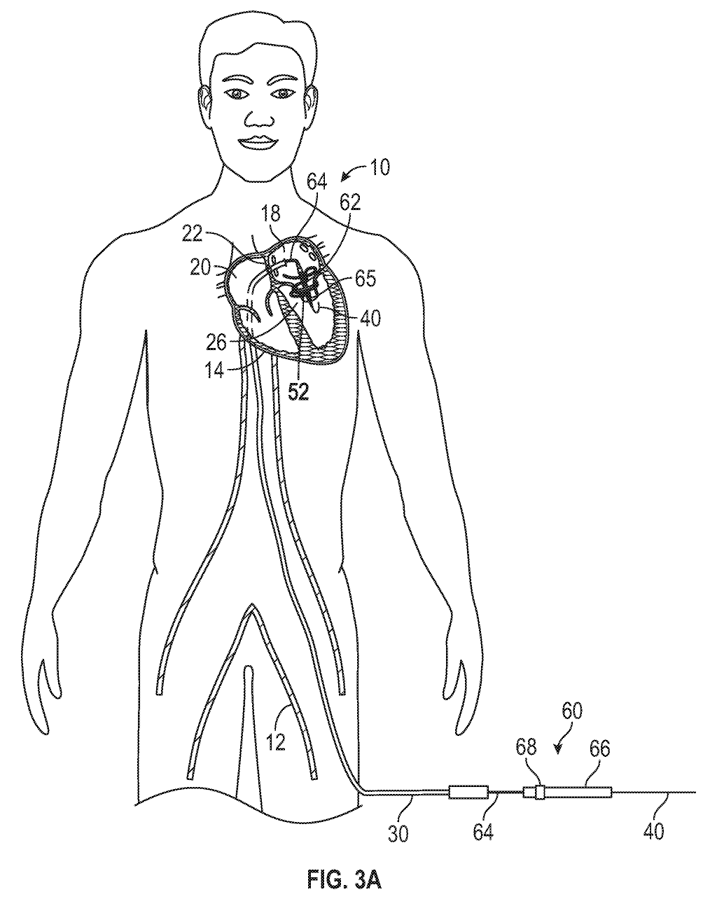

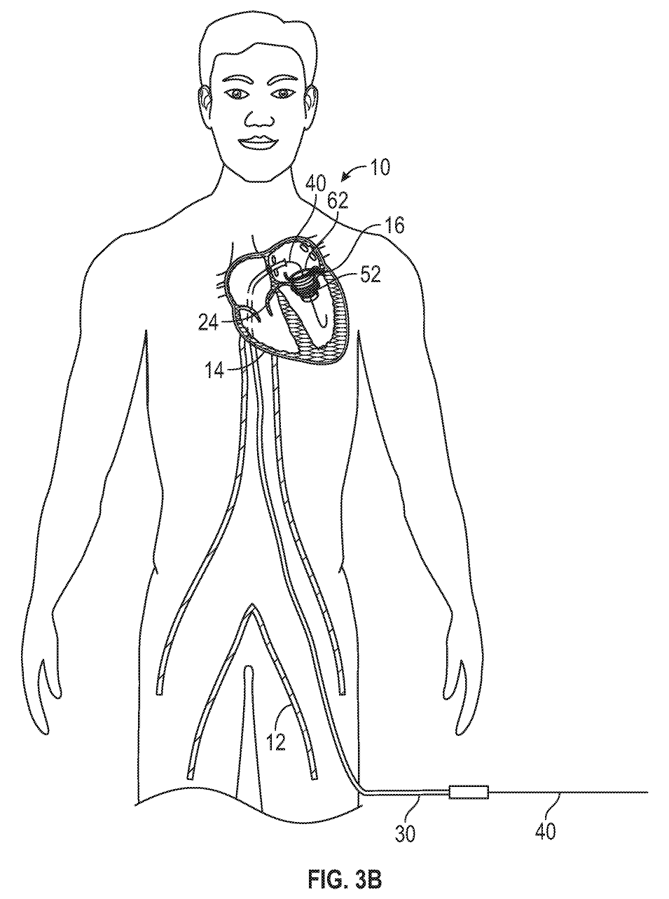

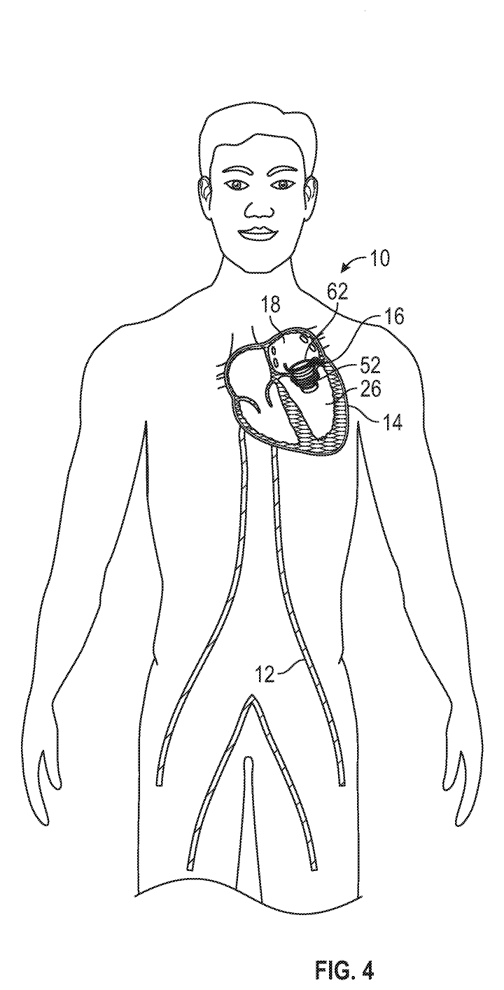

FIGS. 1-4 depict an example of a transcatheter heart valve replacement procedure (such as a mitral valve replacement procedure) which utilizes a docking device 52 and a prosthetic heart valve 62, according to one example. During the procedure, a user first creates a pathway to a patient’s native heart valve using a guide catheter 30 (FIG. 1). The user then delivers and implants the docking device 52 at the patient’s native heart valve using a delivery apparatus 50 (FIG. 2A) and then removes the delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the delivery apparatus 50 and the prosthetic valve delivery apparatus 60 to be navigated through and along, to the implantation site (the native mitral valve 16 or native mitral valve annulus). As shown, the heart 14 is illustrated schematically. For example, the anterior leaflet and chordae of the native mitral valve 16 are omitted for illustration purposes, such that only a portion of the posterior leaflet of the native mitral valve 16 is illustrated.

Initially, the user may first make an incision in the patient’s body to access the blood vessel 12. For example, in the example illustrated in FIG. 1, the user may make an incision in the patient’s groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (such as the delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 (which may also be referred to as a catheter shaft 34) extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (such as the guide catheter 30, the delivery apparatus 50, the prosthetic valve delivery apparatus 60, additional catheters, or the like) and their associated devices (such as docking device, prosthetic heart valve, and the like) to the implantation site within the heart 14, and thus may extend all the way through the blood vessel 12 and into a left atrium 18 of the heart 14 (FIG. 1) and in some examples, through the native mitral valve 16 and into a left ventricle 26 of the heart 14.

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (such as through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts another stage in the example mitral valve replacement procedure where a docking device 52 is being implanted at the native mitral valve 16 of the heart 14 of the patient 10 using a delivery apparatus 50 (which may also be referred to as an “implant catheter,” a dock delivery system,” a “docking device delivery apparatus,” and/or a “docking device delivery device”).

In general, the delivery apparatus 50 comprises a delivery shaft 54 (which may also be referred to as a “dock delivery system shaft”), a handle 56 (which may also be referred to as a “dock delivery system handle”), and a pusher assembly 58. The delivery shaft 54 is configured to be advanced through the patient’s vasculature (blood vessel 12) and to the implantation site (such as native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the delivery apparatus 50 is configured to be gripped and/or otherwise held by the user, outside the body of the patient 10, to advance the delivery shaft 54 through the patient’s vasculature (such as the blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (such as the native mitral valve 16). For example, the pusher assembly 58 is configured to be adjusted by the user to push the docking device 52 out of the distal end portion 53 of the delivery shaft 54. A shaft (which may also be referred to as a “pusher shaft”) of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the delivery apparatus 50 such that the docking device 52 can be released after being deployed at the native mitral valve 16.

Further details of the docking device delivery apparatus and its variants are described in International Publication No. WO2020/247907, PCT Application No. PCT/US2023/18813, and U.S. Provisional Patent Application No 63/380,796, which are incorporated by reference herein in their entirety.

Referring again to FIG. 2A, after the guide catheter 30 is positioned within the left atrium 18, the user may insert the delivery apparatus 50 (such as the delivery shaft 54) into the patient 10 by advancing the delivery shaft 54 of the delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the delivery apparatus 50 through the blood vessel 12 along the guidewire 40 until the delivery shaft 54 reaches the left atrium 18, as illustrated in FIG. 2A. Specifically, the user may advance the delivery shaft 54 of the delivery apparatus 50 by gripping and exerting a force on (for example, by pushing) the handle 56 of the delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (such as the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (such as the portion of the docking device 52 shown in FIG. 2A that is configured to be positioned within the left ventricle 26 and/or on the ventricular side of the native mitral valve 16), the user may then deploy the remaining portion of the docking device 52 (such as an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the delivery apparatus 50 from the docking device 52. Once the docking device 52 is disconnected from the delivery apparatus 50, the user may retract the delivery apparatus 50 out of the blood vessel 12 and away from the patient 10 so that the user can deliver and implant a prosthetic heart valve 62 within the implanted docking device 52 at the native mitral valve 16.

FIG. 2B depicts this stage in the mitral valve replacement procedure, where the docking device 52 has been fully deployed and implanted at the native mitral valve 16 and the delivery apparatus 50 (including the delivery shaft 54) has been removed from the patient 10 such that only the guidewire 40 and the guide catheter 30 remain inside the patient 10. In some examples, after removing the delivery apparatus 50, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

Further details of the docking device and its variants are described in WO 2022/087336, which is incorporated by reference herein in its entirety.

FIG. 3A depicts another stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient’s vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient’s vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (such as the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, by pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, by inflating the inflatable balloon), thereby radially expanding the prosthetic heart valve 62 within the docking device 52.

Further details of the prosthetic heart valve delivery apparatus and its variants are described in WO 2020/247907, which is incorporated by reference herein in its entirety.

Further details of the prosthetic heart valve and its variants are described in U.S. Patent No. 11,185,406, which is incorporated by reference herein in its entirety.

FIG. 3B shows another stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts another stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (such as tricuspid, pulmonary, and/or aortic valves). Further, the same and/or similar delivery apparatuses (such as the delivery apparatus 50, prosthetic valve delivery apparatus 60, guide catheter 30, and/or guidewire 40), docking devices (such as the docking device 52), replacement heart valves (such as the prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient’s vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

### Example Guide Catheter

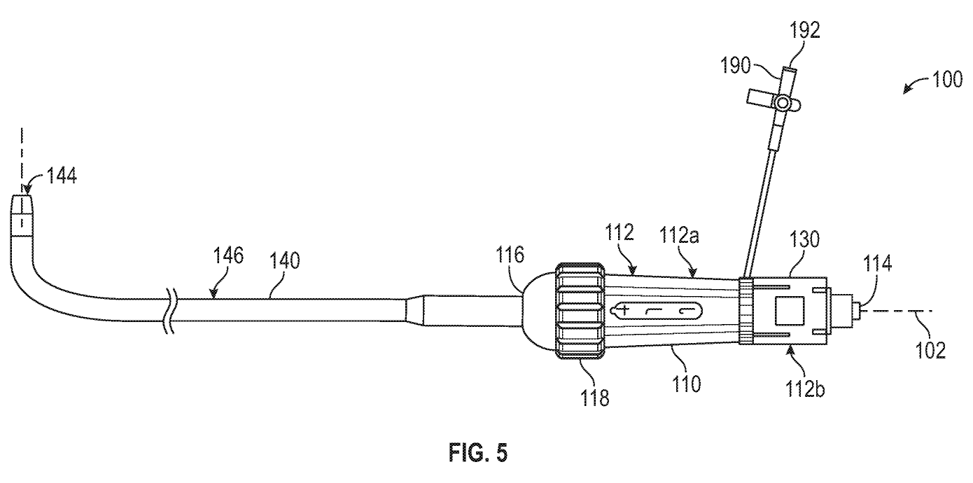

FIG. 5 illustrates a guide catheter, which is referred to below as a guide catheter 100 (but is also referred to herein as a “delivery apparatus” and/or an “introducer device”), according to one example. In some examples, the guide catheter 100 can be used as the guide catheter 30 in a prosthetic valve implantation procedure, such as the procedure described above with reference to FIGS. 1-4. The guide catheter 100 can be configured to be inserted into a patient’s vasculature and receive an implant catheter (and/or other delivery apparatus) therein in order to introduce the implant catheter into the patient’s vasculature and at least partially guide the implant catheter to a target implantation site. The guide catheter 100 can be configured to receive a variety of delivery apparatuses or implant catheters, such as docking device delivery apparatuses, prosthetic heart valve delivery apparatuses, and/or delivery apparatuses for other medical devices or other medical therapies.

The guide catheter 100 in the illustrated example comprises a handle 110, a guide catheter shaft 140, and a central longitudinal axis 102 (which is also referred to as a “longitudinal axis”). The guide catheter shaft 140 can extend proximally into the handle 110.

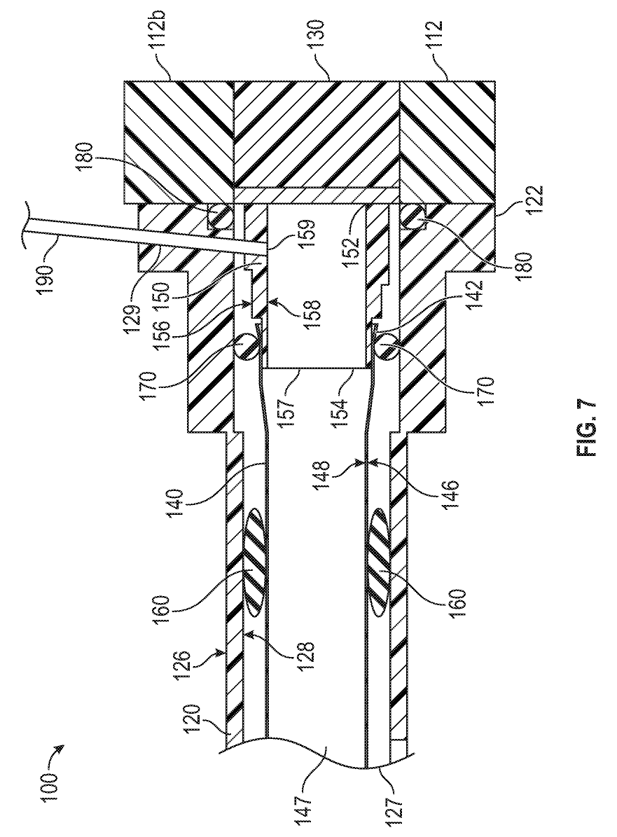

The handle 110 can include a housing 112. In some instances, the housing 112 can be integrally formed as a single, unitary component. In other instances, the housing 112 can comprise one or more segments or portions that are formed as separate components that are coupled together (for example, via fasteners, adhesive, mating features, and/or other means for coupling). As shown, the housing 112 comprises a main segment 112a and a seal stack segment 112b proximally disposed relative to the main segment 112a. The main segment 112a (which is also referred to herein as a “main housing” and/or a “main portion”) can include steering components, as described in more detail below. The seal stack segment 112b (which is also be referred to herein as a “seal stack housing” and/or a “seal stack portion”) of the housing 112 can house a seal stack 130 (FIG. 7), as described in more detail below, therein.

The handle 110 can further comprise a handle lumen (which is also referred to herein as a “lumen”) extending at least partially through the axial length of the handle 110 and/or the housing 112 in a direction parallel to the central longitudinal axis 102. In some examples, the handle lumen can extend from an inlet port 114 disposed at a proximal end of the handle 110, through the handle 110, and to a distal end portion 116 of the handle 110.

The guide catheter shaft 140 (which is also referred to herein as a “guide catheter inner shaft,” “guide sheath inner shaft,” “inner shaft,” and/or “shaft”) can comprise a hollow shaft or tube with a proximal end portion 142 (FIG. 7), a distal end portion 144, a radially outwards-facing outer surface 146, a radially inwards-facing inner surface 148 (FIG. 7), and a guide catheter shaft lumen 147 (FIG. 7) defined by the inner surface 148 and extending through the axial length of the guide catheter shaft 140 from the proximal end portion 142 to the distal end portion 144. The guide catheter shaft lumen 147 can be configured to receive a delivery apparatus and/or a prosthetic medical device therein. For example, the guide catheter shaft lumen 147 can be configured to receive a delivery apparatus and a prosthetic heart valve crimped around a distal end portion of the delivery apparatus.

As introduced above, the main segment 112a of the handle 110 can include a steering mechanism configured to adjust the curvature of the distal end portion 144 of the guide catheter shaft 140 (as such, the guide catheter shaft 140 can be referred to as a “steerable shaft”). In the illustrated example, the handle 110 includes an adjustment member, such as the illustrated rotatable knob 118. The main segment 112a of the housing 112 can house internal flex mechanisms of the guide catheter 100 which are operatively coupled to the rotatable knob 118. In some examples, the flex mechanisms, and thus the knob 118, can be operatively coupled to the proximal end portion of a pull wire. The pull wire can extend distally from the handle 110 through the guide catheter support member 120 and have a distal end portion affixed to the guide catheter shaft 140 (for example, a pull ring of the guide catheter shaft 140) at or near the distal end portion 144 of the guide catheter shaft 140. Rotating the knob 118 can increase or decrease the tension in the pull wire, thereby adjusting the curvature of the distal end portion 144 of the guide catheter shaft 140. Further details on steering or flex mechanisms for a delivery apparatus can be found in U.S. Patent No. 9,339,384, which is incorporated by reference herein in its entirety.

In some instances, the guide catheter shaft 140 can be integrally formed as a single, unitary component. In some instances, the guide catheter shaft 140 can comprise one or more segments that are formed as separate components that are coupled together (for example, via fasteners, adhesive, mating features, and/or other means for coupling). In some examples, a portion of the guide catheter shaft 140 can comprise a material that is more prone to flexing, bending, twisting, etc. than the remaining portion of the guide catheter shaft 140 (for example, a polymer having relatively lower durometer hardness). This can enable the curvature of a portion of the guide catheter shaft 140 to be adjusted or increased at a different rate than the remaining portion of the guide catheter shaft 140 when the pull wires are tensioned. The guide catheter shaft 140 can also include one or more reinforcing braids or jackets that makes the guide catheter shaft 140 more resistant to flexing, bending, twisting, etc., for example, to prevent the guide catheter shaft lumen 147 from kinking or collapsing when the guide catheter shaft 140 is manipulated.

As introduced above, the seal stack 130 (which is also referred to herein as a “seal stack portion”) can be configured to fluidly seal the handle lumen extending through the axial length of the handle 110 from the external environment, while allowing a delivery apparatus (such as any of the prosthetic device delivery apparatuses or implant catheters described herein) to pass therethrough. The seal stack 130 can comprise at least one seal (for example, a plurality of seals) configured to prevent blood from a patient in which the guide catheter 100 is inserted from exiting the guide catheter 100 and prevent air from the environment from entering the guide catheter 100, for example, when the delivery apparatus is inserted into and/or removed from the guide catheter 100 (for example, through the inlet port 114). The at least one seal of the seal stack 130 can include any type of seal, such as a duckbill seal, a flapper seal, an umbrella valve, a cross-slit valve, a disc valve, a dome valve, or the like. As shown, the seal stack 130 and the seal stack segment 112b of the housing 112 are distally adjacent the inlet port 114 and proximally adjacent the main segment 112a of the housing 112.

As shown, the guide catheter 100 comprises an optional flush port 190 extending from the handle 110. The flush port 190 can be fluidly coupled to a lumen (for example, a first adaptor lumen 157 and/or a guide catheter shaft lumen 147, each of which are shown in FIG. 7) of the guide catheter 100. The flush port 190 can be configured to receive fluid through a lumen 192 thereof. In this way, the flush port 190 can be used to maintain and/or improve the lubricity of the lumen of the guide catheter 100, which can allow for improved movement of a delivery apparatus (such as those described herein) relative to the guide catheter 100.

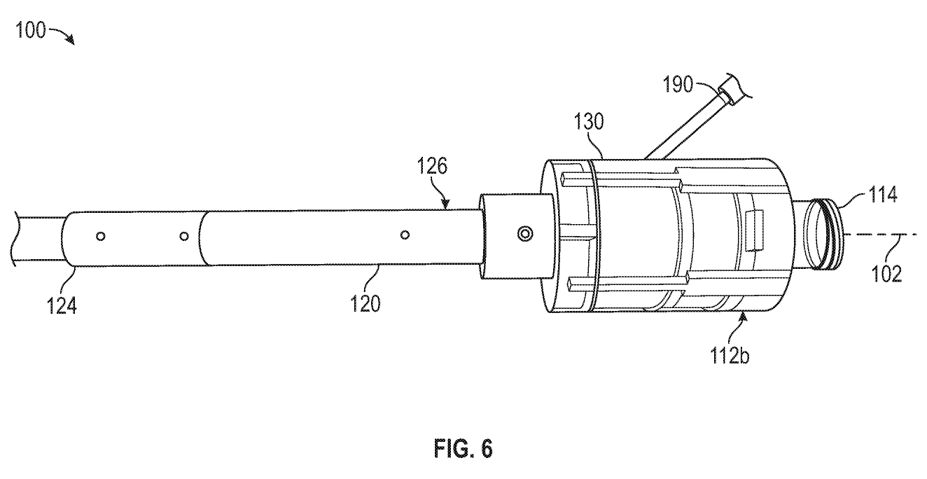

FIG. 6 is a side view of the seal stack segment 112b of the housing 112 and a guide catheter support member 120 coupled thereto, according to one example. FIG. 6 can also be understood as a side view of a proximal portion of the guide catheter 100 with the handle 110 removed. A proximal end portion 122 of the guide catheter support member 120 can be coupled to a distal end portion of the seal stack segment 112b.

The guide catheter support member 120 (which is also referred to herein as a “guide sheath support member,” a “guide catheter spine,” a “guide catheter outer shaft,” a “guide sheath spine,” a “support member,” a “support,” a “guide sheath outer shaft,” an “outer shaft,” and/or a “spine”) can comprise a proximal end portion 122 (FIG. 7), a distal end portion 124, a radially outwards-facing outer surface 126, a radially inwards-facing inner surface 128 (FIG. 7), and a first guide catheter support member lumen 127 (FIG. 7) (which is also referred to herein as a “major guide catheter support member lumen”) defined by the inner surface 128 and extending through the axial length of the guide catheter support member 120 from the proximal end portion 122 to the distal end portion 124. Both the proximal end portion 122 and the distal end portion 124 of the guide catheter support member 120 can be disposed within the handle 110 (for example, within the handle lumen). The guide catheter support member 120 can be configured to circumferentially surround and receive at least a portion of the guide catheter shaft 140 therein. For example, the first guide catheter support member lumen 127 can be configured to receive at least a portion of the guide catheter shaft 140. In some examples, the guide catheter support member 120 can extend through at least a distal portion of the handle lumen. In some examples, the guide catheter support member 120 can be coupled to a proximal portion of the guide catheter 100 (for example, the distal end portion of the seal stack segment 112b).

In some instances, the guide catheter support member 120 can be integrally formed as a single, unitary component. In some instances, the guide catheter support member 120 can comprise one or more segments that are formed as separate components that are coupled together (for example, via fasteners, adhesive, mating features, and/or other means for coupling). In some examples, a portion of the guide catheter support member 120 can comprise a material that is more prone to flexing, bending, twisting, etc. than the remaining portion of the guide catheter support member 120 (for example, a polymer having relatively lower durometer hardness). This can enable the curvature of a portion of the guide catheter support member 120 to be adjusted or increased at a different rate than the remaining portion of the guide catheter support member 120.

FIG. 7 is a cross-sectional view of a proximal end portion of the guide catheter 100, according to one example. As shown, the guide catheter 100 comprises the housing 112 (specifically, the seal stack segment 112b of the housing 112), the guide catheter support member 120 distally disposed relative to the seal stack segment 112b of the housing 112, the seal stack 130 proximally disposed relative to the proximal end portion 122 of the guide catheter support member 120 and disposed within the seal stack segment 112b of the housing 112, the guide catheter shaft 140 disposed at least partially within the guide catheter support member 120, an adaptor 150 coupled to the guide catheter shaft 140, and a sealing member 170 radially disposed between the guide catheter support member 120 and the guide catheter shaft 140. In some examples, the guide catheter 100 can optionally comprise an adhesive portion 160 radially disposed between the guide catheter support member 120 and the guide catheter shaft 140 and distally disposed relative to the adaptor 150. In some examples, the guide catheter 100 can optionally comprise an O-ring 180 axially disposed between the seal stack segment 112b and the guide catheter support member 120. In some examples, the guide catheter 100 can optionally comprise the flush port 190 fluidly coupled to the guide catheter shaft lumen 147 (which is also referred to herein as a “shaft lumen”).

As shown, a distal end portion of the seal stack segment 112b of the housing 112 abuts the proximal end portion 122 of the guide catheter support member 120. These two components can be coupled together to fluidly seal the first guide catheter support member lumen 127 from the external environment. In some examples, the O-ring 180 can be disposed between the seal stack segment 112b and the guide catheter support member 120 to further fluidly seal the first guide catheter support member lumen 127 from the external environment. In some examples, the O-ring 180 can be seated in an annular groove formed at the proximal end portion 122 of the guide catheter support member 120.

As shown, the guide catheter support member 120 further comprises a second guide catheter support member lumen 129 (which is also referred to herein as a “minor guide catheter support member lumen”) extending from the outer surface 126 to the inner surface 128 of the guide catheter support member 120. The second guide catheter support member lumen 129 can allow the first guide catheter support member lumen 127 to be fluidly coupled to the flush port 190.

As shown, the proximal end portion 142 of the guide catheter shaft 140 is flared in the proximal direction of the guide catheter shaft 140. In some examples, it can be easier to insert the adaptor 150 into a flared end of the catheter guide shaft 140 than an unflared end of the catheter guide shaft 140; thus, in such examples, the flared proximal end portion 142 can facilitate the assembly of the guide catheter 100. However, it should be understood that some examples of the catheter guide shaft 140 can have a non-flared proximal end portion 142.

The guide catheter shaft 140 can extend distally through at least a portion of the handle lumen of the handle 110 and/or the first guide catheter support member lumen 127 of the guide catheter support member 120. For example, as shown, the guide catheter shaft 140 can extend through the handle lumen such that the guide catheter support member 120 is disposed circumferentially around the proximal end portion 142 of the guide catheter shaft 140. In some examples, the guide catheter shaft 140 can be wholly disposed within the first guide catheter support member lumen 127. In some examples, the guide catheter shaft 140 can be only partially disposed within the first guide catheter support member lumen 127.

The adaptor 150 can comprise an annular body with a proximal end portion 152, a distal end portion 154, a radially outwards-facing outer surface 156, a radially inwards-facing inner surface 158, and a first adaptor lumen 157 (which is also referred to herein as a “main adaptor lumen”) defined by the inner surface 158 and extending through the axial length of the adaptor 150 from the proximal end portion 152 to the distal end portion 154. The adaptor 150 can be configured to facilitate the coupling and/or connection of the seal stack 130 to the guide catheter shaft 140. In some examples, the adaptor 150 (and/or any adaptor disclosed herein) can be formed from a more rigid material than the guide catheter shaft 140 (and/or any guide catheter shaft disclosed herein). Such a relative difference in rigidity can, in some examples, help provide for improved sealing between the guide catheter shaft 140 and the adaptor 150.

In some examples, the adaptor 150 can further comprise a second adaptor lumen 159 (which is also referred to herein as a “minor adaptor lumen”) extending from the outer surface 156 to the inner surface 158 of the adaptor 150. The second adaptor lumen 159 can allow the first guide catheter support member lumen 127 to be fluidly coupled to the guide catheter shaft lumen 147.

The adaptor 150 can be distally disposed and adjacent the seal stack 130 and at least partially disposed within the proximal end portion 142 of the guide catheter shaft 140. In such examples, the adaptor 150 can be axially aligned with the guide catheter shaft 140 such that at least a portion of the proximal end portion 142 is disposed circumferentially around the adaptor 150 and such that the outer surface 156 of the adaptor 150 contacts the inner surface 148 of the guide catheter shaft 140. In some examples, the adaptor 150 can be axially aligned with the guide catheter support member 120 such that the second guide catheter support member lumen 129 is aligned with the second adaptor lumen 159, thereby allowing the flush port 190 to be fluidly coupled to the first adaptor lumen 157 and/or the guide catheter shaft lumen 147.

The optional adhesive portion 160 can comprise a body of adhesive material configured to fluidly seal a gap formed between the guide catheter support member 120 and the guide catheter shaft 140. The adhesive portion 160 can circumferentially surround the guide catheter shaft 140 and can be radially disposed between the guide catheter support member 120 and the guide catheter shaft 140. As further shown, the adhesive portion 160 is distally disposed relative to the adaptor 150 and distally disposed relative to the proximal end portion 142 of the guide catheter shaft 140. In some examples, the adhesive portion 160 (and/or any adhesive portions disclosed herein) can comprise a UV-cured adhesive.

The sealing member 170 can comprise an annular body configured to circumferentially surround a portion of the proximal end portion 142 of the guide catheter shaft 140. The sealing member 170 can be configured to seal the gap formed between the guide catheter support member 120 and the guide catheter shaft 140, such that the sealing member 170 contacts the outer surface 146 of the guide catheter shaft 140 and the inner surface 128 of the guide catheter support member 120. In some examples, the sealing member 170 (and/or any sealing member disclosed herein) can comprise an O-ring. In some examples, the sealing member 170 (and/or any sealing member disclosed herein) can comprise a molded structure. For example, the sealing member 170 can comprise a structure formed by injecting a mold material (for example, liquid silicone rubber (“LSR”)) into the gap formed between the guide catheter support member 120 and the guide catheter shaft 140. In some examples, the sealing member 170 can beneficially provide for improved sealing between the guide catheter support member 120 and the guide catheter shaft 140, thereby further improving the reliability and robustness of the guide catheter 100.

The sealing member 170 can be axially aligned with the proximal end portion 142 of the guide catheter shaft 140 and the adaptor 150 (for example, the proximal end portion 152 of the adaptor 150), such that the proximal end portion 142 of the guide catheter shaft 140 becomes sandwiched between the adaptor 150 and the sealing member 170. In such examples, the outer surface 156 of the adaptor 150 can support the guide catheter shaft 140 at the location(s) where the sealing member 170 exerts force on the guide catheter shaft 140, thereby preventing the guide catheter shaft 140 from collapsing in a radially inwards direction. In some examples, the sealing member 170 (and/or any sealing member disclosed herein) can be chemically bonded (for example, adhered) to at least one of the guide catheter support member 120 and/or the guide catheter shaft 140. In some examples, the sealing member 170 and/or any sealing member disclosed herein) is not chemically bonded to either the guide catheter support member 120 or the guide catheter shaft 140.

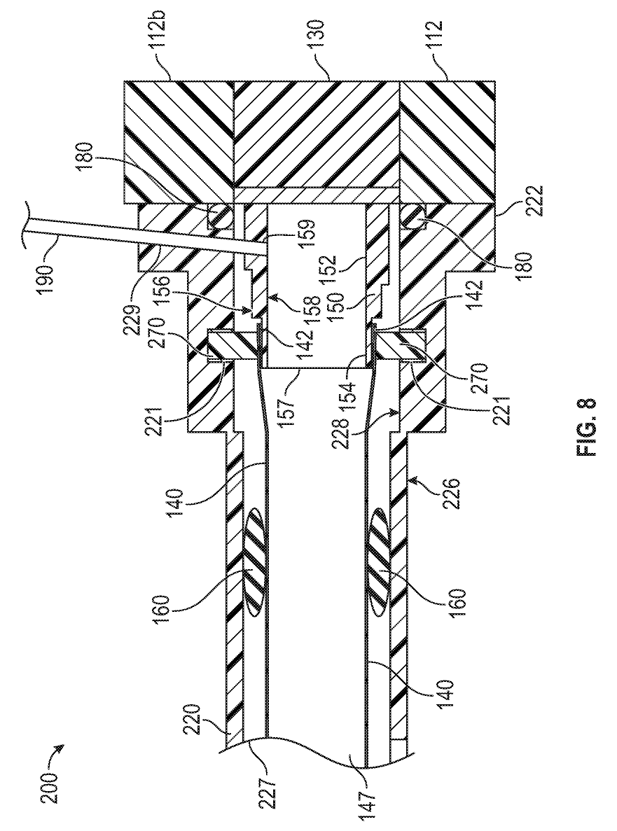

FIG. 8 is a cross-sectional view of a proximal end portion of a guide catheter 200, according to one example. As shown, the guide catheter 200 comprises the seal stack segment 112b of the housing 112, a guide catheter support member 220 distally disposed relative to the seal stack segment 112b, the seal stack 130 proximally disposed relative to a proximal end portion 222 of the guide catheter support member 220 and disposed within the seal stack segment 112b, the guide catheter shaft 140 disposed at least partially within the guide catheter support member 220, the adaptor 150 coupled to the guide catheter shaft 140, and a sealing member 270 radially disposed between the guide catheter support member 220 and the guide catheter shaft 140. In some examples, the guide catheter 200 can optionally comprise the adhesive portion 160 radially disposed between the guide catheter support member 220 and the guide catheter shaft 140 and distally disposed relative to the adaptor 150. In some examples, the guide catheter 200 can optionally comprise the O-ring 180 axially disposed between the seal stack segment 112b and the guide catheter support member 220. In some examples, the guide catheter 200 can optionally comprise the flush port 190 fluidly coupled to the guide catheter shaft lumen 147.

In some examples, some of the components illustrated in FIG. 8 can share similar features with corresponding components illustrated in the previous figures. Such corresponding components are indicated by similar reference numbers (for example, reference numbers incremented by hundreds).

The guide catheter support member 220 can comprise the proximal end portion 222, a distal end portion, a first guide catheter support member lumen 227 extending from the proximal end portion 222 to the distal end portion, an outer surface 226, an inner surface 228, and a second guide catheter support member lumen 229 extending from the outer surface 226 to the inner surface 228.

One exemplary difference between the guide catheter 200 and the guide catheter 100 shown in FIG. 7 is that the guide catheter support member 220 of the guide catheter 200 can comprise a sealing channel 221, wherein the sealing channel 221 can be configured to receive a portion of the sealing member 270 therein. The sealing channel 221 can extend in a radially outwards direction from the inner surface 228 of the guide catheter support member 220 and towards the outer surface 226 of the guide catheter support member 220. Thus, the surfaces of the sealing channel 221 can also be the inner surface 228 of the guide catheter support member 220. The sealing channel 221 can also extend in a circumferential direction along the circumference of the inner surface 228.

As shown, the sealing channel 221 is aligned in the axial direction with the proximal end portion 142 of the guide catheter shaft 140 and with the distal end portion 154 of the adaptor 150. In some examples, the sealing channel 221 can help align the sealing member 270 with the proximal end portion 142 to provide for a better seal.

In this illustrated configuration, the proximal end portion 142 of the guide catheter shaft 140 can be sandwiched between the adaptor 150 and the sealing member 270. The sealing member 270 can be configured to exert compression on both the inner surface 228 of the guide catheter support member 220 and the outer surface 146 of the guide catheter shaft 140 to help fluidly seal the guide catheter shaft lumen 147, thereby helping to prevent the ingress of fluid (for example, air, blood, saline, etc.) into the guide catheter shaft lumen 147.

FIG. 9 is a cross-sectional view of a proximal end portion of a guide catheter 300, according to one example. As shown, the guide catheter 300 comprises the seal stack segment 112b of the housing 112, a guide catheter support member 320 distally disposed relative to the seal stack segment 112b, the seal stack 130 proximally disposed relative to a proximal end portion 322 of the guide catheter support member 320 and disposed within the seal stack segment 112b, the guide catheter shaft 140 disposed at least partially within the guide catheter support member 320, the adaptor 150 coupled to the guide catheter shaft 140, and a sealing member 370 radially disposed between the guide catheter support member 320 and the guide catheter shaft 140. The sealing member 370 can be disposed at least partially within a sealing channel 321 formed in the guide catheter support member 320. In some examples, the guide catheter 300 can optionally comprise the O-ring 180 axially disposed between the seal stack segment 112b and the guide catheter support member 320. In some examples, the guide catheter 200 can optionally comprise the flush port 190 fluidly coupled to the guide catheter shaft lumen 147. Although the presently illustrated guide catheter 300 is shown without the adhesive portion 160, some examples of the guide catheter 300 can comprise the adhesive portion 160.

In some examples, some of the components illustrated in FIG. 9 can share similar features with corresponding components illustrated in the previous figures. Such corresponding components are indicated by similar reference numbers (for example, reference numbers incremented by hundreds).

The guide catheter support member 320 can comprise the proximal end portion 322, a distal end portion, a first guide catheter support member lumen 327 extending from the proximal end portion 322 to the distal end portion, an outer surface 326, an inner surface 328, and a second guide catheter support member lumen 329 extending from the outer surface 326 to the inner surface 328.

One exemplary difference between the guide catheter 300 and the guide catheters 100 and 200 shown in FIGS. 7 and 8 is that the guide catheter support member 320 can comprise a sealing channel 321 with an aperture 323 (which is also referred to herein as a “circumferential aperture”). The sealing channel 321 can extend from the inner surface 328 of the guide catheter support member 320 in a radially outwards direction. Thus, the surfaces of the sealing channel 321 can also be the inner surface 328 of the guide catheter support member 320. The location of the sealing channel 321 and/or the location of the aperture 323 can be aligned in the axial direction with the proximal end portion 142 of the guide catheter shaft 140 and/or with the distal end portion 154 of the adaptor 150. The aperture 323 can be the radially outwards-most portion of the sealing channel 321.

The aperture 323 can be formed in the outer surface 326 of the guide catheter support member 320 and extend partially around the circumference of the outer surface 326. A circumferential portion of the sealing channel 321 can extend up to and/or connect to the aperture 323. For example, the sealing channel 321 can comprise an optional tapered portion 325 that extends up to the aperture 323. As described in more detail below, some examples of the aperture 323 can beneficially facilitate the fabrication of the guide catheter 300—and more specifically the sealing member 370—by allowing mold material to be injected through the aperture 323 to over-mold the sealing member 370 onto the guide catheter support member 320.

The tapered portion 325 is a circumferential portion of the sealing channel 321 that tapers in a radially inwards direction from the aperture 323. As described in more detail below, some examples of the tapered portion 325 can be configured to receive a corresponding locking element 376 (described in more detail below) therein to better secure the sealing member 370 to the guide catheter support member 320. In such examples, the tapered portion 325 and the locking element 376 can be understood to form a keyed joint, wherein the tapered portion 325 can be understood to be a “keyway” and the locking element 376 can be understood to be a “key” of the keyed joint. In such examples, the keyed joint can help provide for improved sealing within the guide catheter 300 by preventing relative movement between the guide catheter support member 320 and the sealing member 370.

Another exemplary difference between the guide catheter 300 shown in FIG. 9 and the guide catheters 100 and 200 shown in FIGS. 7 and 8 is that the illustrated sealing member 370 comprises a proximal annular portion 372, a distal annular portion 374 extending distally from the proximal annular portion 372, and the optional locking element 376 extending in the radially outwards direction from the proximal annular portion 372.

The proximal annular portion 372 can comprise an annular body configured to circumferentially surround at least a portion of the proximal end portion 142 of the guide catheter shaft 140. As shown, the proximal annular portion 372 occupies a portion of the sealing channel 321 because the locking element 376 (not the proximal annular portion 372) occupies the tapered portion 325 of the sealing channel 321. However, in some examples where the sealing channel 321 lacks the tapered portion 325 and the sealing member 370 lacks the locking element 376, the proximal annular portion 372 can occupy the entirety of the sealing channel 321 and can thus extend to the aperture 323 and be flush with the outer surface 326 of the guide catheter support member 320.

As shown, the distal annular portion 374 has a smaller diameter than the proximal annular portion 372.

The locking element 376 can comprise a tapered body extending in the radially outwards direction from the proximal annular portion 372. The taper of the locking element 376 can be complementary to the taper of the tapered portion 325 of the sealing channel 321 such that the tapered portion 325 and the locking element 376 form the keyed joint discussed above. In some examples, the locking element 376 can occupy the entirety of the tapered portion 325. In some examples, the locking element 376 can extend through the tapered portion 325 of the sealing channel 321 to be flush with the outer surface 326 of the guide catheter support member 320.

FIGS. 10-13 depict one example of a portion of a guide catheter fabrication process. More specifically, FIGS. 10-13 depict one example of a process of over-molding the sealing member 370 onto the guide catheter support member 320. The exemplary process is depicted with the guide catheter 300 shown in FIG. 9, but some examples of this process can be used to create any guide catheter or portion of any guide catheter (for example, a guide catheter support member and/or a sealing member) disclosed herein. During the first step of the procedure, the guide catheter support member 320 can be formed (FIG. 10). First and second core pins 410 and 420 can then be inserted into opposing ends of the first guide catheter support member lumen 327 to form a sealing member cavity 430 therein (FIG. 11). Mold material 440 can then be injected through the aperture 323 and into the sealing channel 321 (FIG. 12). The first and second core pins 410 and 420 can then be removed, resulting in the over-molded sealing member 370 (FIG. 13).

FIG. 10 is a cross-sectional view a stage of the guide catheter fabrication process in which the guide catheter support member 320 is formed, according to one example. In some examples, the guide catheter support member 320 can be machined. In such examples, the sealing channel 321 can be undercut. In some examples, the guide catheter support member 320 can be molded. In such examples, molding can beneficially reduce the complexity of forming the guide catheter support member 320 by eliminating the need to undercut the sealing channel 321.

FIG. 11 is a cross-sectional view a stage of the guide catheter fabrication process in which first and second core pins 410 and 420 are inserted into the first guide catheter support member lumen 327, according to one example. Each of the first core pin 410 and the second core pin 420 can comprise a metal rod. As shown, the first core pin 410 comprises a distal end portion 414 with an annular groove 415 formed thereon. As further shown, the second core pin 420 comprises a proximal end portion 422.

As shown, the first core pin 410 is inserted into an axially-facing aperture at the proximal end portion 322 of the guide catheter support member 320 and the second core pin 420 is inserted into an axially-facing aperture at a distal end portion of the guide catheter support member 320. The first core pin 410 can be advanced in the distal direction such that the annular groove 415 aligns with the sealing channel 321. The second core pin 420 can then be advanced in the proximal direction such that the proximal end portion 422 of the second core pin 420 abuts the distal end portion 414 of the first core pin 410.

When the first core pin 410 abuts the second core pin 420, the first core pin 410, the second core pin 420, and the guide catheter support member 320 can form the sealing member cavity 430. The sealing member cavity 430 can comprise an annular cavity in the shape of the sealing member 370. In some examples, the sealing member cavity 430 can comprise a remaining portion of the first guide catheter support member lumen 327 not occupied by the first and second core pins 410 and 420. Since the sealing channel 321 can receive at least a portion of the sealing member 370 therein, the sealing member cavity 430 can be defined at least in part by the sealing channel 321. For example, the aperture 323 in the sealing channel 321 can also be an aperture in the sealing member cavity 430. As described in more detail below, the sealing member cavity 430 can be configured to receive a mold material 440 therein.

FIG. 12 is a cross-sectional view a stage of the guide catheter fabrication process in which mold material 440 is injected into the sealing member cavity 430, according to one example. In some examples, the mold material 440 can comprise liquid silicone rubber (“LSR”). In some examples, the mold material 440 can be injected through the aperture 323 of the sealing channel 321 to completely occupy or fill the sealing member cavity 430.

FIG. 13 is a cross-sectional view a stage of the guide catheter fabrication process in which the first core pin 410 and the second core pin 420 are removed from the first guide catheter support member lumen 327, according to one example, resulting in the over-molded sealing member 370.

FIG. 14 is a cross-sectional view of a proximal end portion of a guide catheter 500, according to one example. One exemplary difference between the guide catheter 500 and the previously described guide catheters is that the guide catheter 500 comprise a sealing member 570. As shown, the sealing member 570 comprises a proximal annular portion 572, a distal annular portion 574, a locking element 576, and a lead-in 578 (which is also referred to herein as a “lead-in surface”). As shown, the lead-in 578 comprises a chamfered surface that tapers in the distal direction. In some examples, the lead-in 578 can beneficially provide for a better seal between the sealing member 570 and a guide catheter shaft (for example, guide catheter shaft 140 and/or any of the guide catheter shafts described herein) by providing a larger sealing surface and by ensuring proper alignment between a shaft, an adaptor, and a sealing member of the guide catheter 500 during the guide catheter assembly process.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus comprising a shaft, a support member, and a sealing member. The shaft extends in an axial direction of the delivery apparatus and comprises an outer surface and a proximal end portion. The support member is disposed circumferentially around the proximal end portion of the shaft and comprises an inner surface and a sealing channel extending radially outwardly from the inner surface. The sealing member is radially disposed between the shaft and the support member and at least partially disposed within the sealing channel, and the sealing member contacts the outer surface of the shaft and the inner surface of the support member.

Example 2. The delivery apparatus of any example herein, particularly Example 1, wherein the sealing member can comprise a locking element.

Example 3. The delivery apparatus of any example herein, particularly Example 2, wherein the locking element can extend in a radially outwards-facing direction.

Example 4. The delivery apparatus of any example herein, particularly any one of Examples 2-3, wherein the locking element can taper in a radially inwards-facing direction.

Example 5. The delivery apparatus of any example herein, particularly any one of Examples 1-4, wherein the sealing member can occupy the entirety of the sealing channel.

Example 6. The delivery apparatus of any example herein, particularly any one of Examples 1-5, wherein the sealing member can comprise a chamfered surface tapering in a distal direction of the delivery apparatus.

Example 7. The delivery apparatus of any example herein, particularly any one of Examples 1-5, wherein the sealing member can comprise a proximal annular portion and a distal annular portion, and wherein the proximal annular portion can have a larger diameter than the distal annular portion.

Example 8. The delivery apparatus of any example herein, particularly any one of Examples 1-7, wherein the sealing member can be a molded component.

Example 9. The delivery apparatus of any example herein, particularly any one of Examples 1-8, wherein the sealing member is not chemically bonded to either the shaft or the support member.

Example 10. A delivery apparatus can include a handle, a handle lumen extending through the axial length of the handle, a shaft extending distally through at least a portion of the handle lumen and axially aligned with the handle lumen, a support member circumferentially surrounding the proximal end portion of the shaft, and a sealing member disposed at least partially within the sealing channel. The shaft can include a shaft lumen extending through the axial length of the shaft and a proximal end portion. The support member can include a sealing channel extending from an inner surface of the support member in a radially outwards-facing direction and a sealing member disposed at least partially within the sealing channel, wherein the sealing member comprises an annular body contacting an outer surface of the shaft at the proximal end portion.

Example 11. The delivery apparatus of any example herein, particularly Example 10, wherein the sealing channel can further comprise a circumferential aperture formed on an outer surface of the support member.

Example 12. The delivery apparatus of any example herein, particularly any one of Examples 10-11, wherein the sealing channel can comprise a tapered portion that tapers in a radially inwards-facing direction.

Example 13. The delivery apparatus of any example herein, particularly Example 12, wherein the sealing member can comprise a locking element, and wherein the tapered portion can be configured to receive the locking element therein.

Example 14. The delivery apparatus of any example herein, particularly Example 13, wherein the locking element can be tapered, and wherein the taper of the locking element can be complementary to the taper of the tapered portion.

Example 15. The delivery apparatus of any example herein, particularly any one of Examples 10-14, which can further comprise an adaptor, wherein the proximal end portion of the shaft circumferentially can surround and at least partially overlap the adaptor.

Example 16. The delivery apparatus of any example herein, particularly Example 15, wherein the adaptor is formed from a more rigid material than the shaft.

Example 17. The delivery apparatus of any example herein, particularly any one of Examples 15-16, wherein the sealing channel can be aligned in an axial direction with the proximal end portion of the shaft and a distal end portion of the adaptor.

Example 18. The delivery apparatus of any example herein, particularly any one of Examples 10-17, wherein the proximal end portion of the shaft can be flared.

Example 19. The delivery apparatus of any example herein, particularly any one of Examples 10-18, which can further comprise an adhesive portion coupled to the outer surface of the shaft and the inner surface of the support member, wherein the adhesive portion can be distally disposed relative to the sealing member.

Example 20. A method of forming a delivery apparatus can include forming a support member comprising a support member lumen extending through the axial length of the support member and a sealing channel extending from an inner surface of the support member. The method can further include inserting a first core pin into a first end of the support member lumen, axially aligning a first end portion of the first core pin with the sealing channel, inserting a second core pin into a second end of the support member lumen, axially aligning a second end portion of the second core pin with the first end portion of the first core pin, and injecting a mold material into the sealing channel to form a sealing member.

Example 21. The method of any example herein, particularly Example 20, wherein forming the support member can include molding the support member.

Example 22. The method of any example herein, particularly any one of Examples 20-21, wherein the sealing channel can comprise a circumferential aperture formed in the support member, and wherein the mold material can be injected through the circumferential aperture of the support member into the sealing channel.

Example 23. The method of any one of claims 20-22 wherein the mold material can be liquid silicone rubber (LSR).

Example 24. The delivery apparatus of any example herein, wherein the delivery apparatus can be sterilized.

The features described herein with regard to any example can be combined with other features described in any one or more of the other examples, unless otherwise stated. For example, any one or more of the features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A delivery apparatus comprising:
a shaft extending in an axial direction of the delivery apparatus, wherein the shaft comprises an outer surface and a proximal end portion;
a support member disposed circumferentially around the proximal end portion of the shaft, wherein the support member comprises an inner surface and a sealing channel extending radially outwardly from the inner surface; and
a sealing member radially disposed between the shaft and the support member and at least partially disposed within the sealing channel, wherein the sealing member contacts the outer surface of the shaft and the inner surface of the support member.

2. The delivery apparatus of claim 1, wherein the sealing member comprises a locking element.

3. The delivery apparatus of claim 2, wherein the locking element extends in a radially outwards-facing direction.

4. The delivery apparatus of claim 2, wherein the locking element tapers in a radially inwards-facing direction.

5. The delivery apparatus of claim 1, wherein the sealing member occupies the entirety of the sealing channel.

6. The delivery apparatus of claim 1, wherein the sealing member comprises a chamfered surface tapering in a distal direction of the delivery apparatus.

7. The delivery apparatus of claim 1, wherein the sealing member comprises a proximal annular portion and a distal annular portion, wherein the proximal annular portion has a larger diameter than the distal annular portion.

8. A delivery apparatus comprising:
a handle;
a handle lumen extending through the axial length of the handle;
a shaft extending distally through at least a portion of the handle lumen and axially aligned with the handle lumen, the shaft comprising:
a shaft lumen extending through the axial length of the shaft; and
a proximal end portion;
a support member circumferentially surrounding the proximal end portion of the shaft, the support member comprising:
a sealing channel extending from an inner surface of the support member in a radially outwards-facing direction; and
a sealing member disposed at least partially within the sealing channel, wherein the sealing member comprises an annular body contacting an outer surface of the shaft at the proximal end portion.

9. The delivery apparatus of claim 8, wherein the sealing channel further comprises a circumferential aperture formed on an outer surface of the support member.

10. The delivery apparatus of claim 8, wherein the sealing channel comprises a tapered portion that tapers in a radially inwards-facing direction.

11. The delivery apparatus of claim 10, wherein the sealing member comprises a locking element, and wherein the tapered portion is configured to receive the locking element therein.

12. The delivery apparatus of claim 11, wherein the locking element is tapered, and wherein the taper of the locking element is complementary to the taper of the tapered portion.

13. The delivery apparatus of claim 8, further comprising an adaptor, wherein the proximal end portion of the shaft circumferentially surrounds and at least partially overlaps the adaptor.

14. The delivery apparatus of claim 13, wherein the adaptor is formed from a more rigid material than the shaft.

15. The delivery apparatus of claim 13, wherein the sealing channel is aligned in an axial direction with the proximal end portion of the shaft and a distal end portion of the adaptor.

16. The delivery apparatus of claim 8, wherein the proximal end portion of the shaft is flared.

17. The delivery apparatus of claim 8, further comprising an adhesive portion coupled to the outer surface of the shaft and the inner surface of the support member, wherein the adhesive portion is distally disposed relative to the sealing member.

18. A method of forming a delivery apparatus comprising:
forming a support member comprising:
a support member lumen extending through the axial length of the support member; and
a sealing channel extending from an inner surface of the support member;
inserting a first core pin into a first end of the support member lumen;
axially aligning a first end portion of the first core pin with the sealing channel;
inserting a second core pin into a second end of the support member lumen;
axially aligning a second end portion of the second core pin with the first end portion of the first core pin; and
injecting a mold material into the sealing channel to form a sealing member.

19. The method of claim 18, wherein forming the support member includes molding the support member.

20. The method of claim 18, wherein:
the sealing channel comprises a circumferential aperture formed in the support member, and
the mold material is injected through the circumferential aperture of the support member into the sealing channel.
